(19) United States
(12) Patent Application Publication
(10) Pub. No.: US 2007/0068515 A1
(43) Pub. Date: Mar. 29, 2007

(54) Apparatus for administration of aromatherapy on a massage table or chair

(76) Inventor: Natalie Churchill, Ladera Ranch, CA (US)

Correspondence Address: Ramon M. Valencia, 7 Ambrose, Aliso Viejo, CA, 92656, US

(21) Appl. No.: 11/524,095

(22) Filed: Sep. 20, 2006

Related U.S. Application Data
(60) Provisional application No. 60/720,104, filed on Sep. 23, 2005.

(51) Int. Cl. A61K 36/18; A61M 11/00

(52) U.S. Cl. 128/200.23; 424/725

(57) ABSTRACT

The present invention relates to devices for providing aromatherapy to a person undergoing massage therapy. More specifically, but without restriction to the particular embodiments hereinafter described in accordance with the best mode of practice, this invention relates to aroma devices for providing aromatherapy while undergoing massage therapy and methods of making and using the aromatherapy device of the present invention. A preferred embodiment of the aroma device of the present invention comprises an aroma container, a clamp, and a connector joining the aroma container to the clamp. The connector may be formed from a flexible material allowing movement of the aroma container relative to the clamp. The aroma container and clamp may be coupled to the connector by a ball and socket joint, a snap-fit joint, welding, adhesive, screws, bolts or threading.

### CROSS-REFERENCE TO RELATED APPLICATIONS

The present application claims the benefit of priority from U.S. Provisional Application No. 60/720,104 filed Sep. 23, 2005 the contents of which is hereby incorporated by reference in its entirety as if fully repeated herein.

### FIELD OF THE INVENTION

This invention relates in general to aromatherapy. More specifically, but without restriction to the particular embodiments hereinafter described in accordance with the best mode of practice, this invention relates to devices for providing aromatherapy while undergoing massage therapy.

### GENERAL DISCUSSION AND RELATED ART

Prior hereto, aromatherapy has been administered by placing therapeutic oils on absorbent materials which are placed near ones face for inhalation. Normally, these inhaled oils are placed on a tissue paper, paper towel, paper napkin, handkerchief or similar absorbent material that allows the oil to evaporate. The oil filled absorbent material is then placed under the pillow of the face cradle of a massage table or massage chair so the individual undergoing a massage may inhale the scent while undergoing a massage.

More particularly, the arts related hereto have been contributed by several inventors including Hagiwara, et al., and MacDonald. As an illustration of such contributors, there is a stress dissolving refreshment system disclosed in U.S. Pat. No. 5,024,650 to Hagiwara, et al. The stress dissolving refreshment system causes a relaxed state induction means actuated during a relaxing period, thereafter a refresh stimulus means actuated to generate a weak stimulation during a disillusion period in which the level of consciousness of the user is gradually elevated, and then the refresh stimulus means further actuated to generate a strong stimulation during a refreshing period in which the level of the user's consciousness is elevated to a level good enough for allowing the user to return to normal working, whereby the consciousness level of the user is quickly elevated after being relaxed, so as to effectively attain the refreshment.

In another patent to Hagiwara, et al., U.S. Pat. No. 5,266,070, there is described a relaxation refreshment apparatus that includes a relaxation refreshment chair provided for giving to the user a vibratory stimulus variable in accordance with a detected relaxing degree of the user, an optical stimulus with exterior light shielded at the top part of the chair, a pneumatic stimulus operatively connected to an aromatizer nearby, for giving to the user an aromatic stimulus and an acoustic stimulus with a sound output unit, the apparatus being thus capable of intensively providing to the user various stimuli with an arrangement simplified and effectively realizing a compactness with dimensional minimization.

In the disclosure of U.S. Patent Application Publication No. 2004/0172064 in the name of Bonnie MacDonald there is described a treatment table having a raised surface portioned to receive a person desiring treatment. The raised surface has an associated face position, either included in the surface or a face rest removably attached to the table for receiving the person's face when the person desiring treatment lays on their stomach on the raised surface. The face position having an opening to facilitate unobstructed breathing. An aroma dispenser is positioned near the opening so that the aroma given off by the dispenser may be inhaled by the person desiring treatment.

What is needed is a simple, adjustable, effective and economical aromatherapy administration device which can be attached to a massage table or chair. It is, therefore, an object of the present invention to provide an improved aromatherapy administering device that can be attached to a massage table or chair and can be appropriately place at a desired distance from the face of a person undergoing massage and aromatherapy.

### BACKGROUND OF THE INVENTION

Aromatherapy can be defined as the art and science of utilizing naturally extracted aromatic essences from plants to balance, harmonize and promote the health of body, mind and spirit. It is an art and science which seeks to explore the physiological, psychological and spiritual realm of the individual's response to aromatic extracts as well as to observe and enhance the individual's innate healing process. As a holistic medicine, aromatherapy is both a preventative approach as well as an active treatment during acute and chronic stages of illness or disease.

It is a natural, non-invasive treatment system designed to affect the whole person not just the symptom or disease and to assist the body's natural ability to balance, regulate, heal and maintain itself by the correct use of essential oils or aromatherapy agents.

Origin of the Word Aromatherapie

The term “aromatherapie” was coined by Rene Maurice Gattefosse in 1928. He utilized the word to imply the therapeutic use of aromatic substances (essential oils). Since the beginning of Aromatherapy, the practice has encompassed human pathology and the treatment of different conditions (emotional and physical) with essential oils. As aromatherapy developed into a practice it adopted a holistic approach which encompasses the body, the mind and the spirit (energy). As a holistic therapy, aromatherapy is able to work on several levels of the individuals well being.

Aromatherapy Massage Applications

In massage therapy applications, an aromatherapy agent, aromatherapy oil or essential oil is scented oil that is applied on skin and is also administered by placing the therapeutic oils on absorbent materials which are placed near ones face for inhalation. Normally, these inhaled oils are placed on a tissue paper, paper towel, paper napkin, handkerchief or similar absorbent material that allows the oil to evaporate. The oil filled absorbent material is then placed under the pillow of the face cradle on a massage table or massage chair so the individual may inhale the scent during a massage. The oils applied to the skin are absorbed through the skin while the inhaled scents enter ones body through the respiratory system. These oils are used to achieve a desired mood or medicinal purposes.

Aromatherapy oils are volatile plant oils, including essential oils, for psychological and physical well-being. An essential oil is a liquid that is generally distilled (most frequently by steam or water) from the leaves, stems, flowers, bark, roots, or other elements of a plant. Essential oils, contrary to the use of the word “oil” are not really oily-feeling at all. Most essential oils are clear, but some oils such as patchouli, orange and lemongrass are amber or yellow in color. Essential oils contain the true essence of the plant it was derived from. Essential oils are highly concentrated and a little goes a long way.

Essential oils are not the same as perfume or fragrance oils. Where essential oils are derived from the true plants, perfume oils are artificially created fragrances or contain artificial substances and do not offer the therapeutic benefits that essential oils offer. The chemical composition and aroma of essential oils can provide valuable psychological and physical therapeutic benefits. These benefits are usually achieved through methods including inhalation and application of the diluted oil to the skin as described above.

Essential oils are often used by diluting them with a carrier oil (sweet almond oil, apricot kernel oil, grapeseed oil) and then applying this blend to the skin for absorption.

Careful inhalation of the oils can also provide therapeutic benefit as the oil molecules enter the lungs and are absorbed into the bloodstream. It is thus an object of the present invention to provide a device that allows proper positioning of an essential oil for optimal inhalation while undergoing a massage without making one uncomfortable in the process.

Essential oils can very greatly in quality and price. Various factors that can affect the quality and price of the oil include the rarity of the plant, country and conditions that the plant was grown, quality standards of the distiller, and how much oil is produced by the plant.

Essential oils can often be purchased as blends of several essential oils. The advantage if it is a blend of pure essential oils is that you can save from having to buy every essential oil individually. The disadvantage is that you have no control over the blend by not mixing it yourself nor can you reliably mix the blend with other oils.

Examples of aromatherapy agents or essential oils that may be used in conjunction with the present invention include, but are not limited to, the aromatherapy agents listed below in Tables 1 and 2:

TABLE 1
Aromatherapy Agents Alphabetized by Common Name:

Angelica Root
Anise
Balsam, Peru
Basil
Bay
Bay Laurel
Beeswax
Benzoin
Bergamot
Bergamot Mint
Bois-de-rose
Boronia
Cajeput
Cardamum
Carrot Seed
Cedarwood, Atlas
Cedarwood, Virginian
Chamomile, German
Chamomile, Roman
Cinnamon
Citronella
Clary Sage
Clove Bud
Coriander
Cypress
Dill
Elemi
Eucalyptus
Eucalyptus, Lemon
Eucalyptus Radiata
Fennel
Fir Needle
Frankincense
Galbanum
Geranium
Geranium, Rose
Ginger
Grapefruit
Helichrysum
Hyssop
Immortelle
Jasmine
Juniper Berry
Kanuka
Lavender
Lavendin
Lemon
Lemongrass
Lime
Linden Blossom
Mandarin
Manuka
Marjoram
May Chang
Myrrh
Myrtle
Myrtle, Lemon
Neroli
Niaouli
Nutmeg
Oakmoss
Olibanum
Orange, Bitter
Orange, Sweet
Oregano
Palmarosa
Parsley
Patchouli
Pepper, Black
Peppermint
Petitgrain
Pine, Scotch
Ravensara
Rose
Rosemary
Rosewood
Sandalwood
Spearmint
Spikenard
Spruce
Tagetes
Tangerine
Tea Tree, Common
Tea Tree, Lemon
Tea Tree, New Zealand
Thyme
Tobacco
Tuberose
Vanilla
Vetiver
Violet Leaf
Yarrow
Ylang Ylang

TABLE 2
Aromatherapy Agents Alphabetized by Botanical Name:

Abies alba
Achillea millefolium
Anethum graveolens
Angelica archangelica
Aniba rosaeodora
Anthemis nobilis
Apis mellifera
Backhousia citriodora
Boronia megastigma
Boswellia carterii
Cananga odorata
Canarium luzonicum
Cedrus atlantica
Cinnamomum zeylanicum
Citrus aurantifolia
Citrus aurantium
Citrus aurantium
Citrus aurantium
Citrus bergamia
Citrus limon
Citrus paradisi
Citrus reticulata
Citrus reticulata
Citrus sinensis
Commiphora myrrha
Coriandrum sativum
Cupressus sempervirens
Cymbopogon citratus
Cymbopogon martini
Cymbopogon nardus
Daucus carota
Elettaria cardamomum
Eucalyptus citriodora
Eucalyptus globulus
Eucalyptus radiata
Eugenia caryophyllata
Evernia prunastri
Ferula galbaniflua
Foeniculum vulgare
Helichrysum angustifolia
Hyssopus officinalis
Jasminum officinalis
Juniperus communis
Juniperus virginiana
Laurus Nobilis
Lavandula hybrida
Lavendula officinalis
Leptospermum ericoides
Leptospermum petersonii
Leptospermum scoparium
Litsea cubeba
Matricaria chamomilla
Melaleuca alternifolia
Melaleuca leucadendron
Melaleuca quinquenervia
Mentha Citrata
Mentha piperita
Mentha spicata
Myristica fragrans
Myroxylon pereirae
Myrtus communis
Nardostachys jatamansi
Nicotania tabacum
Ocimum basilicum
Oreganum majorana
Oreganum vulgare
Pelargonium graveolens
Pelargonium roseum
Petroselinum sativum
Picea mariana
Pimenta racemosa
Pimpinella anisum
Pinus sylvestris
Piper nigrum
Pogostemon cablin
Polianthes tuberosa
Ravensara aromatica
Rosa damascena
Rosmarinus officinalis
Salvia sclarea
Santalum album
Styrax tonkinensis
Tagetes minuta
Thymus vulgaris
Tilia vulgaris
Vanilla planifolia
Vetiveria zizanoides
Viola odorata
Zingiber officinale

### BRIEF DESCRIPTION OF THE INVENTION

The present invention is directed to an apparatus for administration of aromatherapy on a massage table or massage chair. More particularly this invention is directed to a device for providing aromatherapy during a massage when a person is faced down on a massage table or massage chair with his head on a face cradle to achieve optimal inhalation of the therapeutic scent or aromatherapy agent.

Administering aromatherapy during a massage when a person is face down is difficult. Massage therapists currently administer the essential oils by placing it on an absorbent material like a tissue paper and inserting said tissue between the pillow and base plate of a face cradle on a massage table or massage chair. The problem with this current method for administering aromatherapy is that it does not provide enough of the therapeutic oil/scent due to its relative proximity to the person's nose from the source of the aromatherapy.

The current invention provides a solution to the problem by providing a device for easy and convenient directed administration of volatile essential oils. The device of the present invention is an aromatherapy holder that places the aromatherapy close to the person's nose to achieve proper inhalation of the essential oil during an aromatherapy massage. This device is designed to work on virtually all massage tables and massage chairs with a face cradle.

Applicant has therefore discovered a system and method for administering aromatherapy during a massage that substantially eliminates the problems with the current methods.

The above described and many other features and attendant advantages of the present invention will become better understood by reference to the following detailed description when taken in conjunction with the accompanying figures.

### BRIEF DESCRIPTION OF THE FIGURES

Further objects of the present invention together with additional features contributing thereto and advantages accruing therefrom will be apparent from the following description of preferred embodiments of the present invention which are shown in the accompanying figures with like reference numerals indicating like components throughout, wherein:

FIG. 1 is a side view of one embodiment of the aromatherapy apparatus, aromatherapy device or aroma device of the present invention showing the various components of the apparatus including a clamp, a connector, an aroma container and an optional fastener;

FIG. 2 is a top perspective view of the aroma device of FIG. 1;

FIG. 3 is an exploded view of the aroma device of FIG. 1;

FIG. 4 is an illustration of a massage table including a face cradle and base support member or frame such as a base plate;

FIG. 5 is an illustration of a massage chair including a face cradle;

FIG. 6 shows the aroma device of FIGS. 1 to 3 attached to the base plate of a face cradle on a massage table;

FIG. 7 illustrates a person undergoing aromatherapy using the aroma device of FIGS. 1 to 3;

FIGS. 8A to 8F depicts another embodiment of the aroma device of the present invention having a moldable or flexible connector which allows for the movement of the aroma container for optimal positioning;

FIG. 9 illustrates yet another embodiment of the aroma device of the present invention having one form of a “c-type” clamp for attachment of the aroma device to rounded face cradle support members.; and

FIG. 10 shows still another embodiment of the aroma device of the present invention having a round type or rounded clamp.

### DETAILED DESCRIPTION OF THE INVENTION

The present invention is directed to devices for providing aromatherapy while undergoing massage therapy discussed below in further detail.

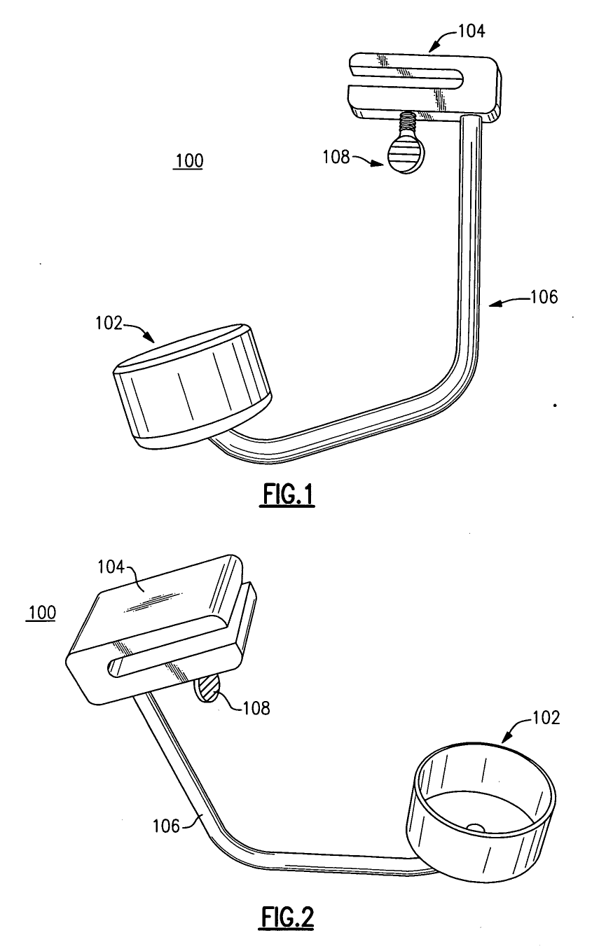

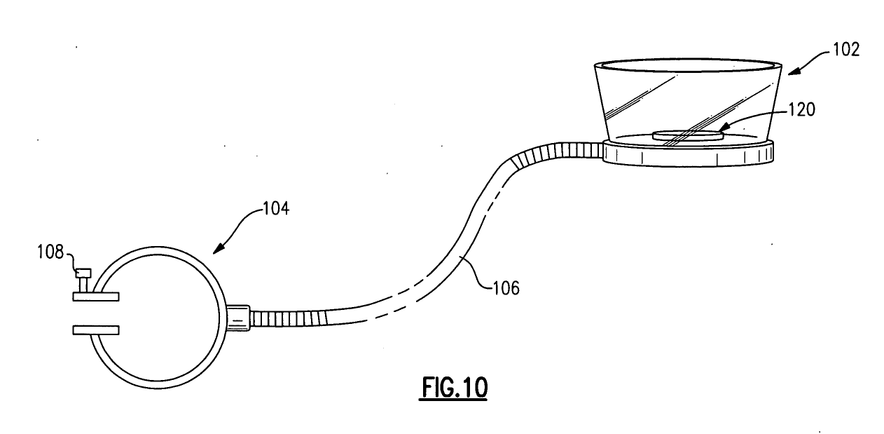

FIG. 1 is a side view depiction of a preferred embodiment of the aromatherapy apparatus, aroma device or aromatherapy device 100 of the present invention. The aroma device includes an aromatherapy agent holder or aroma container 102, a clamp 104 and a connector 106. The aroma container 102 is attached to the clamp 104 through the connector 106. The connector 106 may be attached to the aroma container 102 or the clamp 106 by conventional attachment methods including, but not limited to, ball and socket joints to allow movement of the various components of the device (as described below in conjunction with FIG. 9), snap-fit joint, glued on, welded on, bolted on, screwed on, threaded on (as illustrated in FIG. 3) or any other methods of attachments used in the arts of making plastic or metal devices. Examples of snap-fit attachments or joints are described and illustrated by Paul R. Bonenberger in his book entitled “First Snap-Fit Handbook—Creating Attachments for Plastic Parts”, published by Hanser Publishers which is incorporated by reference in its entirety as if fully repeated herein. Bonenbergers's book discusses snap-fit mechanisms, locators, locating schemes and overconstraints, design-for-assembly, and the snap-fit function from the end user point-of-view. Alternatively, the entire device may be made using a mold, injection molded, for example, in the case of plastics or machined as a single piece in the case of metals, for example.

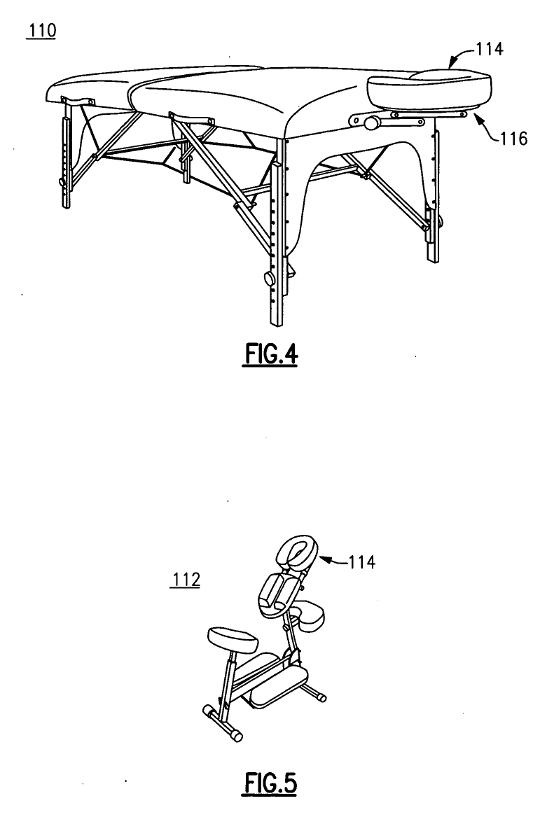

The aroma device 100 may optionally include a fastener 108 to aid in fastening said aroma device onto a face cradle of a massage table or massage chair. The aroma device of the present invention is preferably attached to a face cradle support such as a base plate or any supporting member of the face cradle. An example of a massage table and chair is described below in conjunction with FIGS. 4 and 5, respectively showing a face cradle and a face cradle support or supporting member.

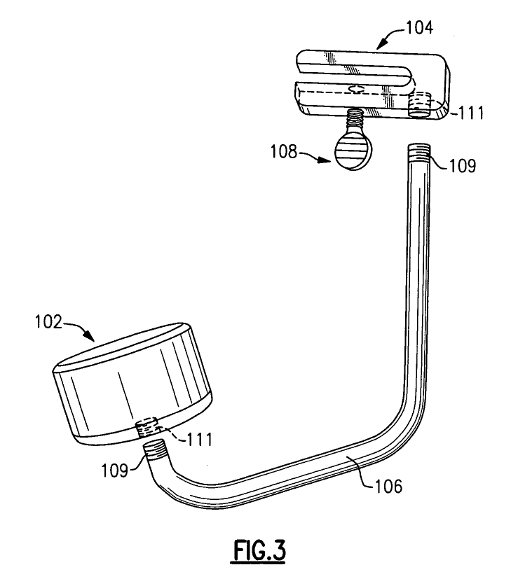

The next figure, FIG. 2 is top-side view of the aroma device 100 of FIG. 1 that shows the “cup-like” aroma container 102 of the aroma device 100. The aroma container 102 is not limited to cup-like holders but may be any shape that holds an absorbent material or liquid. Likewise, the clamp 104 illustrated in FIGS. 1 and 2 is not limited to flat clamps but may include any type of clamp that can be used to attach the aroma device 100 of the present invention to any part of a massage table or chair. These clamps may include, for example, but are not limited to, sliding clamps, C-type clamps, toggle clamps, spring clamps, spring and plier clamps, bar clamps, pipe clamps, and magnetic clamps. An embodiment of the aroma device of the present invention having an alternative clamp is best depicted and described below in conjunction with FIG. 9 and FIG. 10. The various components of the aroma device 100 of FIG. 1 is further depicted in the exploded view of the aroma device 100 shown is the next figure, FIG. 3. There is also illustrated in FIG. 3 threaded attachment points including the male threaded member 109 and the female threaded member 111 for attaching the connector 106 to the aroma container 102 and also to the clamp 104.

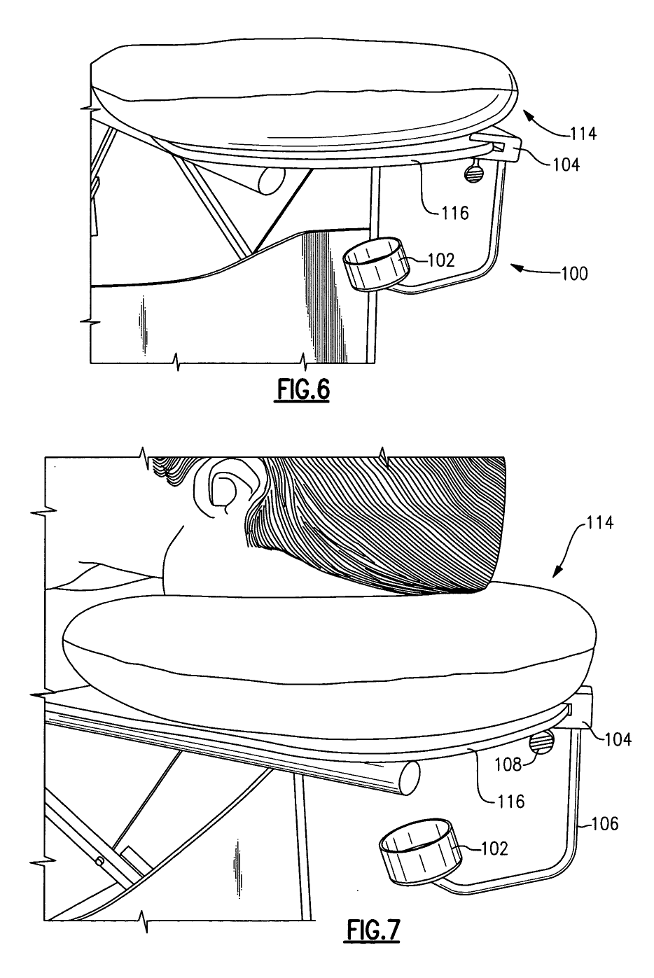

Moving on to the next two figures, FIGS. 4 and 5 show a massage table 110 and a massage chair 112, respectively. The massage table 110 has a face cradle 114 attached to the table through a base plate 116. The aroma device 100 may thus be attached to the base plate 116 to optimally place the aromatherapy agent near ones nose to provide proper inhalation of said aromatherapy agent as illustrated in FIG. 6. As shown in FIG. 6, the aroma device 100 is attached to the base plate 116 using the clamp 104.

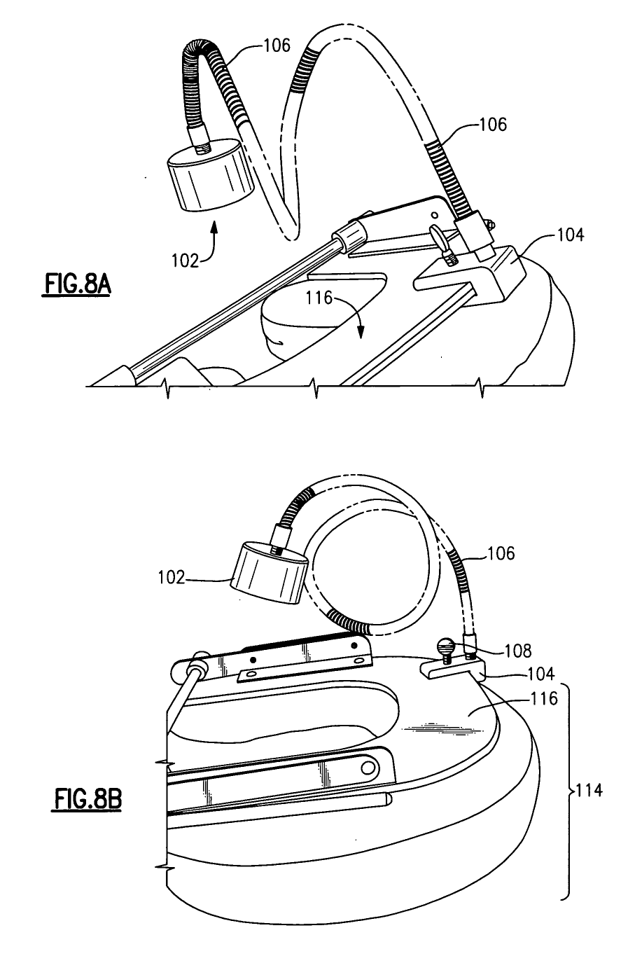

The optimal placement of the aromatherapy agent using the aroma device 100 of the present invention is best illustrated in FIG. 7 which shows a person undergoing massage and aromatherapy using the aroma device 100 of the present invention which places the source of the aromatherapy agent near ones nose for optimal inhalations of said aromatherapy agent.

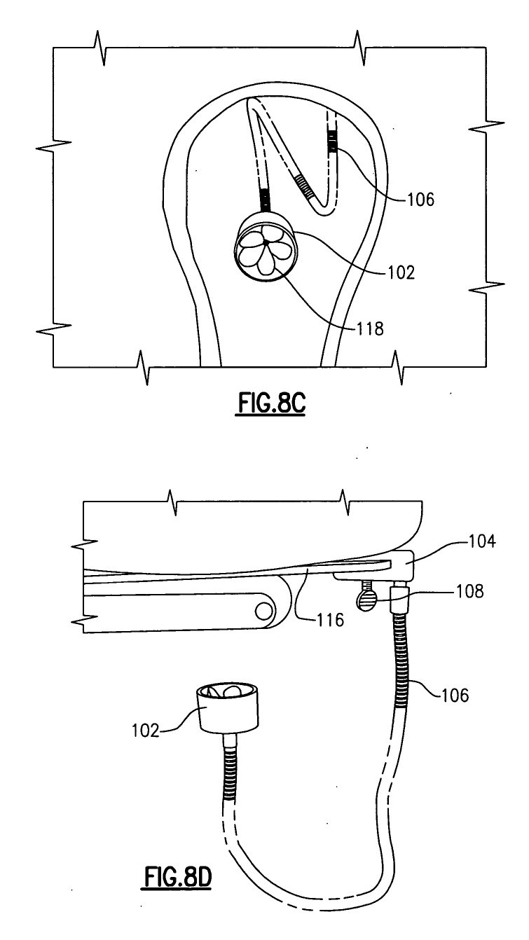

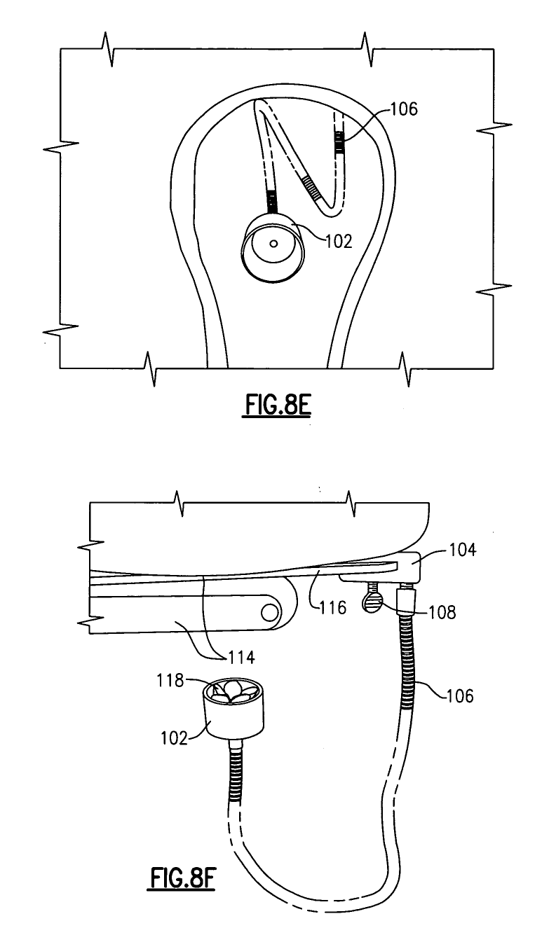

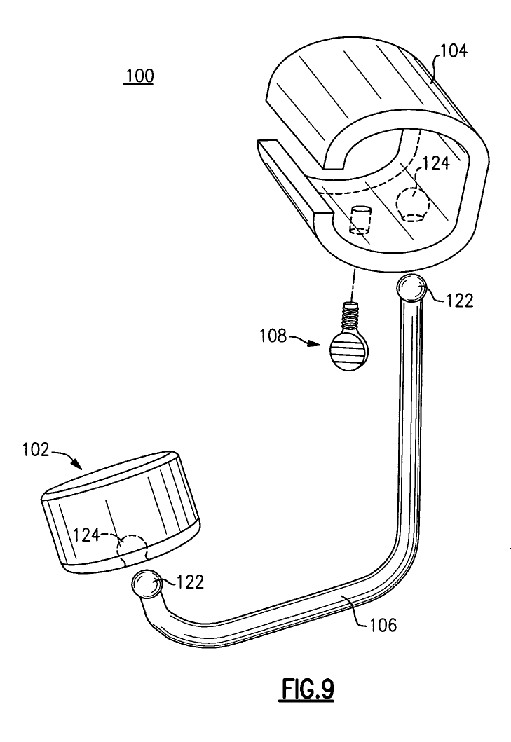

With reference now to FIG. 8, there is illustrated another proffered embodiment of the aroma device of the present invention. The same basic components are also depicted here including the clamp 104, connector 106 and aroma container 102. The aroma container 102 is also “cup-like” as in the device described above in conjunction with FIGS. 1 to 3. The aroma device 100 of FIG. 8 shows an alternative connector 106 which is a flexible material that allows for the customization of the placement of the aroma container relative to ones face, nose and field of view. The flexible material may be formed from a number of flexible materials that retain their shape after it is bent to a desired position including, for example, but not limited to, any flexible metals or flexible plastic materials. With specific reference to FIGS. 8A and 8B showing the alternative device of the present invention attached to the base plate or base member 116 of a face cradle 114. The base member 116 of the face cradle illustrated in FIGS. 8A and 8B is an example of a base plate 116 which is a flat support member usually made of wood. The next figure, FIG. 8C, shows a view of the aroma device 100 from the perspective of a person on a massage table or chair. The aroma container 102 of the device 100 is shown to be close of the persons face or within ones field of view. As shown, a flower 118 may be used as a source of an aroma or an aroma agent. This also illustrates an alternative use of the aroma device for ornamental purposes to entertain and relax an individual during a massage. Often people having a massage complain that they get bored because they stare at the floor the whole time. This way, a massage therapist can place different visually entertaining and stimulating objects in the aroma container 102 to entertain their client. Of course, the therapist can combine both and place an aroma agent and visually stimulating objects in the aroma container of the device of the present invention to simultaneously entertain and provide aromatherapy during a massage. Moving next to FIG. 8D which depicts another configuration of the flexible connector that allows customized placement of the aroma container relative to a persons face and field of view. The next figure, FIG. 8E shows yet another view of the aroma device 100 from the perspective of a person on a massage table or chair without an ornamental piece or object. While FIG. 8F illustrates the aroma container 102 positioned away from the face cradle 114. Since the connector 106 depicted in FIG. 8 is flexible, the therapist can easily move the aroma container 102 out of the way to place or replace an aroma agent and/or ornament in the aroma container 102. The therapist can also conveniently move the aroma container 102 having an aroma agent away from the client, if the client chooses to stop aromatherapy during a massage due to the flexible nature of the connector.

Alternatively, the aroma device 100 of the present invention may be directly attached, built into, or made a part of a massage table or massage chair. As a non-limiting example, of this alternative embodiment of the aroma device 100 of the present invention, one end of the connector 106 of the aroma device may be directly connected to the support or base member 116 of the face cradle such that it becomes part of the face cradle 114 while the other end of the connector 106 is connected to the aroma container 102. Since the connector depicted in FIG. 8 is flexible, the aroma container may be placed or positioned appropriately, as described above. The end of the connector 106 that is attached to the massage table may be attached by various means or methods including, but not limited to, for example, using appropriate fasteners such as nuts and bolts, and hook and loop fasteners, and using appropriate adhesives or welding the connector onto any portion of the massage table or chair.

Referring next to FIG. 9, there is shown an exploded view of yet another embodiment of the present invention having a “c-type” clamp 104 for attachment of the aroma device 100 to rounded face cradle support members or any rounded attachment point on a massage table or chair. Also shown in FIG. 9 is a ball member 122 and a socket member 124 of a ball and socket joint. The ball and socket joint also allow movement of the aroma container which allows the therapist to place the aroma source at a desired location for optimal inhalation of the aroma agent and also allow the therapist to conveniently move the aroma container 102 away from the client, if the client chooses to stop aromatherapy during a massage due to the movable nature of the device with ball and socket joints. The last figure, FIG. 10 illustrates still another embodiment of the aroma device of the present invention having a “round-type” clamp 104 for attachment onto rounded components of a massage table or chair. An aroma source 120 is also depicted in FIG. 10.

### CONCLUDING SUMMARY

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope including, for example, use of the device of the present invention for ornamental purpose such as display of colorful beads and flowers to entertain and relax a person during a massage or making the aromatherapy device an integral part of a massage table or chair.

Furthermore, those skilled in the art will recognize, or be able to ascertain, using no more than routine experimentation, many equivalents to the specific embodiments of the invention described herein. Such equivalents are intended to be encompassed by the following claims.

What is claimed is:

1. An aroma device for administering aromatherapy to a person undergoing massage therapy comprising:
an aroma container for holding an aromatherapy agent;
a clamp; and
a connector having a first end and a second end; said aroma container attached to said first end of said connector; and said clamp attached to said second end of said connector.

2. The aroma device of claim 1 wherein said connector is formed from a flexible material allowing movement of said aroma container relative to said clamp.

3. The aroma device of claim 1 wherein said aroma container and said clamp are attached to said connector through a ball and socket joint, a snap-fit joint, by welding, using an adhesive, a screw, a bolt, or a threaded joint.

4. The aroma device of claim 3 further comprising a fastener to fasten said clamp onto a massage table or chair.

5. A method of using said aroma device of claim 4 comprising the steps of:
placing an aromatherapy agent into said aroma container; and
attaching said aroma device to a massage table or massage chair such that said aroma container is optimally placed to provide proper inhalation of said aromatherapy agent by a person undergoing massage and aromatherapy.

6. A method of making an aroma device for administering aromatherapy to a person undergoing massage therapy comprising the steps of:
forming an aroma container having a first socket of a first ball and socket joint formed therein;
forming a clamp having a second socket of a second ball and socket joint formed therein;
forming a connector having a first end and a second end;
forming a first ball member on said first end of said connector;
forming a second ball member on said second end of said connector;
attaching said first ball member of said connector to said first socket of said aroma container; and
attaching said second ball member of said connector to said second socket of said clamp.

7. A method of using said aroma device of made according to the method of claim 7 for ornamental purposes comprising the steps of:
placing an ornament in said aroma container; and
attaching said aroma device to a massage table or massage chair such that said aroma container is optimally placed within the field of view of the person undergoing a massage.

8. A method of using said aroma device of made according to claim 7 comprising the steps of:
placing an aromatherapy agent in said aroma container;
attaching said aroma device to a massage table or massage chair through said clamp; and
positioning said aroma container containing said aromatherapy agent optimally to provide proper inhalation of said aromatherapy agent by a person undergoing massage and aromatherapy.

9. A method of making an aroma device for administering aromatherapy to a person undergoing massage therapy comprising the steps of:
forming an aroma container;
forming a connector having a first end and a second end;
forming a clamp;
attaching said aroma container to said first end of said connector; and
attaching said clamp to said second end of said connector.

10. The method of claim 9 wherein said connector is formed from a flexible material.

11. The method of claim 10 further comprising the steps of:
forming a first male threaded member of a first threaded joint on said first end of said connector;
forming a second male threaded member of a second threaded joint on said second end of said connector;
forming a first female threaded member of said first threaded joint in said aroma container;
forming a second female threaded member of said second threaded joint in said clamp;
attaching said first female threaded member of said aroma holder to said first male threaded member of said connector by threading in said first male threaded member into said first female threaded member of said first threaded joint; and
attaching said second female threaded member of said clamp to said second male threaded member of said connector by threading in said second male threaded member into said second female threaded member of said second threaded joint.

12. The method of claim 10 further comprising the steps of:
forming a first male member of a first snap-fit joint on said first end of said connector;
forming a second male member of a second snap-fit joint on said second end of said connector;
forming a first female member of said first snap-fit joint in said aroma container;
forming a second female member of said second snap-fit joint in said clamp;
attaching said first female member of said aroma holder to said first male member of said connector by snapping-in said first male member into said first female member; and
attaching said second female member of said clamp to said second male member of said connector by snapping-in said second male member into said second female member.
